(19) United States
(12) Patent Application Publication
(10) Pub. No.: US 2012/0266884 A1
(43) Pub. Date: Oct. 25, 2012

(54) EXHAUST PORT ASSEMBLY THAT MINIMIZES NOISE

(75) Inventors: Peter Chi Fai Ho, Pittsburgh, PA (US); Elizabeth Powell Margaria, Pittsburgh, PA (US)

(73) Assignee: KONINKLIJKE PHILIPS ELECTRONICS N.V., EINDHOVEN (NL)

(21) Appl. No.: 13/518,088

(22) PCT Filed: Nov. 17, 2010
(86) PCT No.: PCT/IB10/55229

§ 371 Date: Jun. 21, 2012

Related U.S. Application Data
(60) Provisional application No. 61/290,340, filed on Dec. 28, 2009.

(51) Int. Cl. A61M 16/06

(52) U.S. Cl. 128/205.25

(57) ABSTRACT

A deflector mechanism (20) for use with a respiratory interface device (8) is provided. The interface device includes a cushion (8a), a mask frame (8b) supporting the cushion, a patient circuit (6) coupled to the mask frame adapted to carry a flow of gas, and an exhaust port (10) integrated in the frame and/or the patient circuit for venting exhaust gases (12) from the respiratory interface device. The deflector mechanism includes a deflector portion (24) having a surface (25) and a mounting portion (22) that couples the a deflector to the frame and/or patient circuit such that vented exhaust gases contact the surface of the deflector portion. The deflector portion is structured to modify sound created by the venting exhaust gases. The deflector portion is also structured to selectively direct the exhaust gases without an addition additional restriction, i.e., a greater pressure drop, to the flow of the exhaust gases.

This patent application claims the priority benefit under 35 U.S.C. §119(e) of U.S. Provisional Application No. 61/290,340 filed on Dec. 28, 2009, the contents of which are herein incorporated by reference.

The present invention relates to the control of flow patterns of fluids, such as gases, and, more particularly, to an apparatus for selectively manipulating the air flow pattern of exhalation gases that may be employed in, for example, a respiratory patient interface device.

It is well known to treat a patient with a non-invasive positive pressure support therapy, in which a flow of breathing gas is delivered to the airway of a patient at a pressure greater than the ambient atmospheric pressure. For example, it is known to use a continuous positive airway pressure (CPAP) device to supply a constant positive pressure to the airway of a patient throughout the patient's respiratory cycle to treat obstructive sleep apnea (OSA), as well as other cardio-pulmonary disorders, such as congestive heart failure (CHF) and Cheynes-Stokes respiration (CSR). Examples of such CPAP devices include the REMstar® family of CPAP devices manufactured by Philips Respironics, Inc. of Murrysville, Pa.

A “bi-level” non-invasive positive pressure therapy, in which the pressure of gas delivered to the patient varies with the patient's breathing cycle, is also known. Such a bi-level pressure support system provides an inspiratory positive airway pressure (IPAP) that is greater than an expiratory positive airway pressure (EPAP). IPAP refers to the pressure of the flow of gas delivered to the patient's airway during the inspiratory phase; whereas EPAP refers to the pressure of the flow of gas delivered to the patient's airway during the expiratory phase. Such a bi-level mode of pressure support is provided by the BiPAP® family of devices manufactured and distributed by Phillips Respironics, Inc. and is taught, for example, in U.S. Pat. Nos. 5,148,802 to Sanders et al., 5,313,937 to Zdrojkowski et al., 5,433,193 to Sanders et al., 5,632,269 to Zdrojkowski et al., 5,803,065 to Zdrojkowski et al., and 6,029,664 to Zdrojkowski et al., the contents of each of which are incorporated herein by reference.

Auto-titration positive pressure therapy is also known. With auto-titration positive pressure therapy, the pressure of the flow of breathing gas provided to the patient changes based on the detected conditions of the patient, such as whether the patient is snoring or experiencing an apnea, hypopnea, or upper airway resistance. An example of a device that adjusts the pressure delivered to the patient based on whether or not the patient is snoring is the Virtuoso® CPAP family of devices manufactured and distributed by Respironics, Inc. This auto-titration pressure support mode is taught, for example, in U.S. Pat. Nos. 5,203,343; 5,458,137 and 6,087,747 all to Axe et al., the contents of which are incorporated herein by reference.

A further example of an auto-titration pressure support device that actively tests the patient's airway to determine whether obstruction, complete or partial, could occur and adjusts the pressure output to avoid this result is the Tranquility® Auto CPAP device, also manufactured by Respironics, Inc. This auto-titration pressure support mode is taught in U.S. Pat. No. 5,645,053 to Remmers et al., the content of which is also incorporated herein by reference.

Other modes of providing positive pressure support to a patient are known. For example, a proportional assist ventilation (PAV®) mode of pressure support provides a positive pressure therapy in which the pressure of gas delivered to the patient varies with the patient's breathing effort to increase the comfort to the patient. U.S. Pat. Nos. 5,044,362 and 5,107,830 both to Younes, the contents of which are incorporated herein by reference, teach a pressure support device capable of operating in a PAV® mode. Proportional positive airway pressure (PPAP) devices deliver breathing gas to the patient based on the flow generated by the patient. U.S. Pat. Nos. 5,535,738; 5,794,615; and 6,105,573 all to Estes et al., the contents of each of which are incorporated herein by reference, teach a pressure support device capable of operating in a PPAP mode.

For purposes of the present invention, the phrases “pressure support device”, “pressure generating device”, and/or “pressure generator” (used interchangeably herein) refer to any medical device adapted for delivering a flow of breathing gas to the airway of a patient, including a ventilator, CPAP, PAV®, PPAP, or bi-level pressure support device. The phrases “pressure support system” and/or “positive pressure support system” (used interchangeably herein) include any arrangement or method employing a pressure support device and adapted for delivering a flow of breathing gas to the airway of a patient.

In a conventional pressure support system a flexible conduit couples the pressure support device to a patient interface device. The flexible conduit forms part of what is typically referred to as a “patient circuit” that carries the flow of breathing gas from the pressure support device to patient interface device. The patient interface device connects the patient circuit with the airway of the patient so that the flow of breathing gas is delivered to the patient's airway. Examples of patient interface devices include a nasal mask, nasal and oral mask, full face mask, nasal cannula, oral mouthpiece, tracheal tube, endotracheal tube, or hood.

In a non-invasive pressure support system, a single-limb patient circuit is typically used to communicate the flow of breathing gas to the airway of the patient. An exhaust port (also referred to as an exhalation vent, exhalation port, and/or exhaust vent) is provided in the patient circuit and/or the patient interface device to allow exhaust gas, such as the exhaled gas from the patient, to vent to atmosphere.

A variety of exhalation ports are known for venting gas from a single-limb patient circuit. For example, U.S. Pat. No. Re. 35,339 to Rappoport discloses a CPAP pressure support system wherein a few exhaust ports are provided directly on the patient interface device, i.e., in the wall of the mask. However, such exhaust port configuration results in a relatively direct stream of exhaust gas being directed from the mask or patient circuit. Direct streaming of the flow of exhaust gas is undesirable, because a typical CPAP system is intended to be used while the patient is asleep. Sleep for the patient or the patient's bed partner is disturbed if a stream of gas is directed at the patient or at the patient's bed partner.

The exhaust port assembly described in U.S. Patent Application Pub. No. 2007/0101998 to Kwok is directed to minimizing the noise associated with the venting of exhaust gases from a respiratory mask. This is allegedly accomplished by providing an elastomeric material around the perimeter of the exhaust port. Such exhaust port configuration, however, does not solve the problem of preventing a generally direct or concentrated stream of gas from being directed from the mask onto the patient or the patient's sleep partner and does not lend itself to being used with existing exhaust ports.

U.S. Pat. No. 5,937,851 to Serowski et al., U.S. Pat. No. 6,112,745 to Lang, and U.S. Pat. No. 6,691,707 to Gunaratnam et al. all disclose exhalation ports for a positive pressure support system. Each of the exhalation ports taught by these references attempts to solve the problem of preventing a stream of gas from being directed onto the patient or onto the patient's bed partner by controlling the direction of the flow of exhaust gas. For example, each of these references teaches directing the flow of gas back along the patient circuit rather than directly outward away from the patient. However, the relative direction of the stream of gas flow changes each time the patient assumes a new sleeping position, and depending on the positioning of the patient circuit, the stream of concentrated gas may be directed onto the patient or the patient's sleep partner.

Accordingly, it is an object of the present invention to provide a pressures support system and method that overcomes the shortcomings of conventional systems. This object is achieved according to one embodiment of the present invention by providing a deflector mechanism for use with a respiratory interface device. The respiratory interface device includes a cushion, a mask frame supporting the cushion, a patient circuit coupled to the mask frame adapted to carry a flow of gas, and an exhaust port integrated in one of the mask frame and patient circuit for venting exhaust gases from the respiratory interface device. The deflector mechanism includes a deflector portion having a surface and a mounting portion. The mounting portion is structured to couple the deflector mechanism to the one of the mask frame and patient circuit at or near the exhaust port such that vented exhaust gases contact the surface of the deflector portion. The deflector portion has a characteristic that is structured to modify sound created by the venting exhaust gases and the deflector portion being further structured to selectively direct the exhaust gases without restricting the flow of the exhaust gases.

In another embodiment, a respiratory interface device is provided. The respiratory interface device includes a cushion, a mask frame supporting the cushion, a patient circuit coupled to the mask frame adapted to carry a flow of gas, an exhaust port integrated in one of the mask frame or patient circuit. The exhaust port is structured to vent a flow of exhaust gases from the respiratory interface device, and a deflector mechanism. The deflector mechanism includes a deflector portion having a surface and a mounting portion coupling the deflector mechanism to the one of the mask frame and patient circuit at or near the exhaust port. The surface of the deflector portion is structured to selectively direct the flow of exhaust gases without restricting the flow of the exhaust gases and the deflector portion includes a characteristic that is structured to modify sound created by the exhaust gases.

In a further embodiment, a method of handling the flow of exhalation gases vented from an exhalation port of a respiratory interface device is provided. The respiratory interface device includes a cushion, a mask frame supporting the cushion, a patient circuit coupled to the mask frame adapted to carry a flow of gas, and an exhaust port integrated in one of the mask frame and patient circuit for venting exhaust gases from the respiratory interface device. The method including providing a deflector mechanism having a deflector portion having a surface and a mounting portion. The deflector portion has a characteristic that is structured to modify sound created by the venting exhaust gases. The method further includes coupling the deflector mechanism via the mounting portion to the one of the mask frame and patient circuit at or near the exhalation port such that vented exhaust gases contact the surface of the deflector portion.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

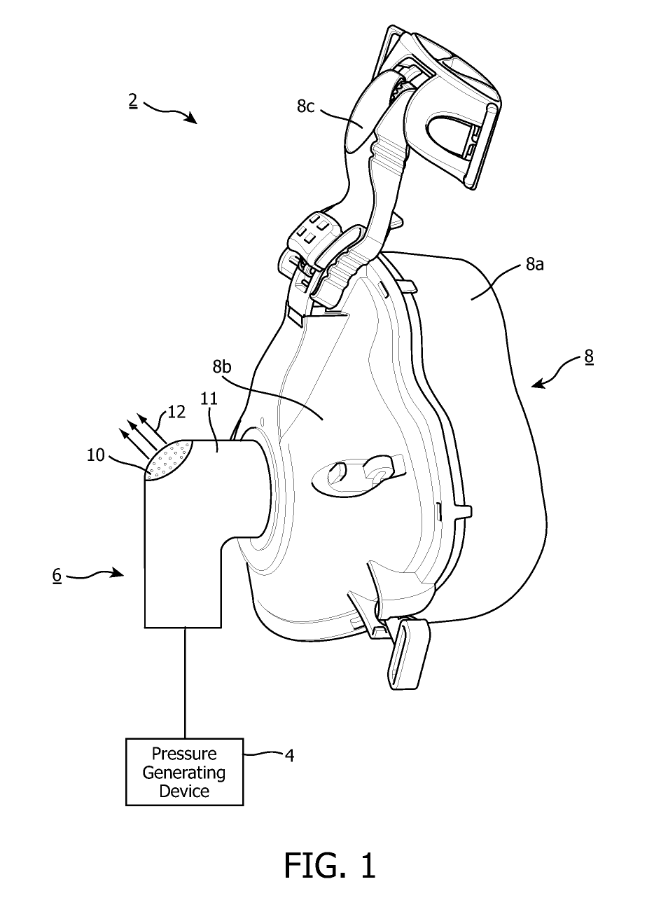

FIG. 1 is a schematic diagram of a known pressure support system adapted to provide a regimen of respiratory therapy to a patient;

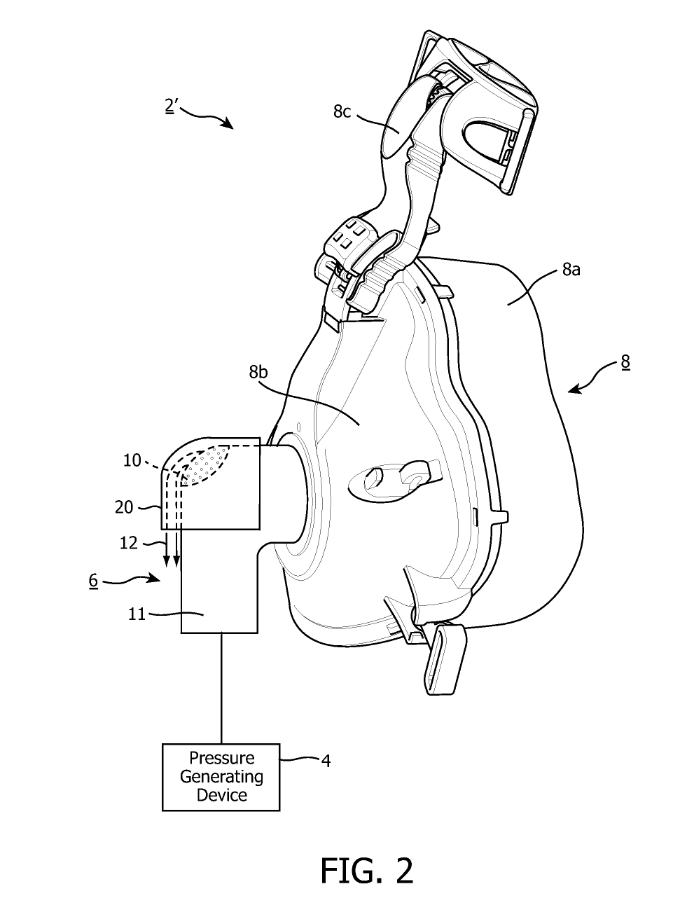

FIG. 2 is a schematic diagram of the pressure support system of FIG. 1 fitted with a deflector mechanism according to one embodiment of the present invention;

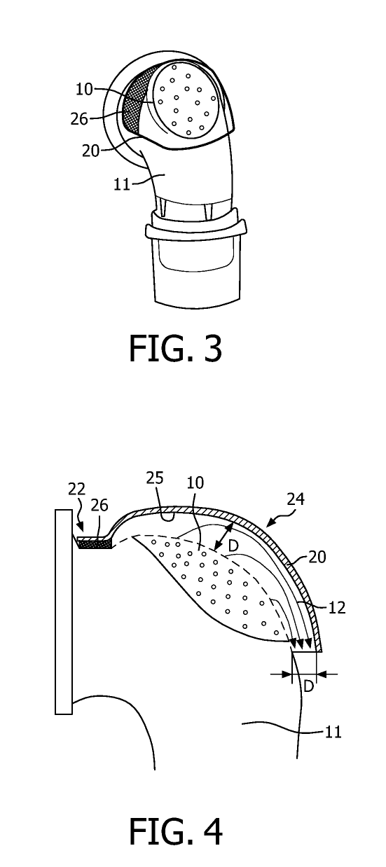

FIG. 3 is an isometric view of a portion of the pressure support system of FIG. 2 including the deflector mechanism;

FIG. 4 is a close-up elevational side view of the portion of FIG. 3 showing additional details of the deflector mechanism;

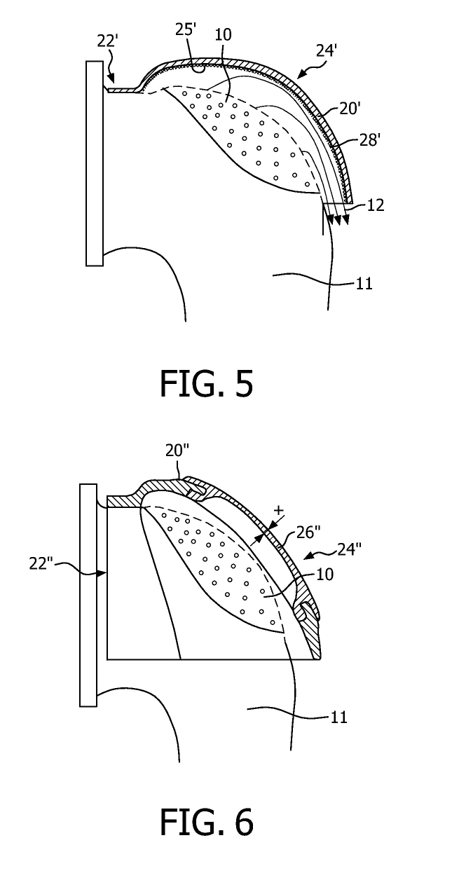

FIG. 5 is a close-up elevational side cross-sectional view of a deflector mechanism in accordance with another embodiment of the invention;

FIG. 6 is a close-up elevational side cross-sectional view of a deflector mechanism in accordance with a further embodiment of the invention;

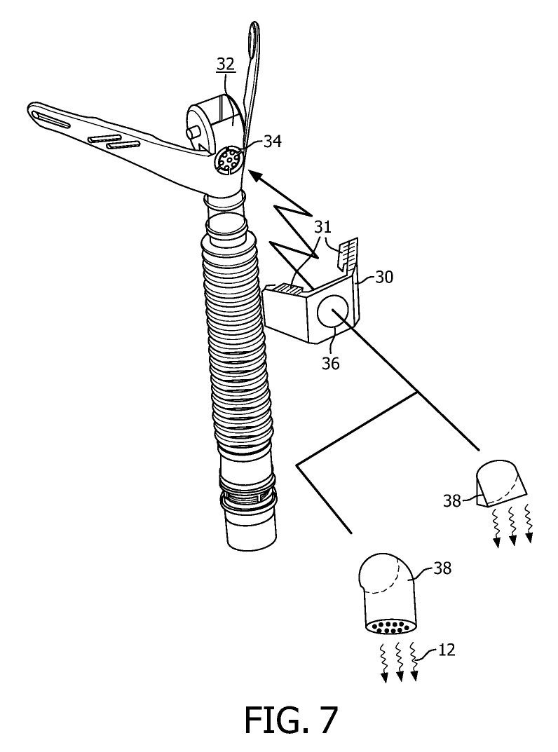

FIG. 7 is an exploded view of another deflector mechanism and a portion of a patient interface device in accordance with an embodiment of the invention; and

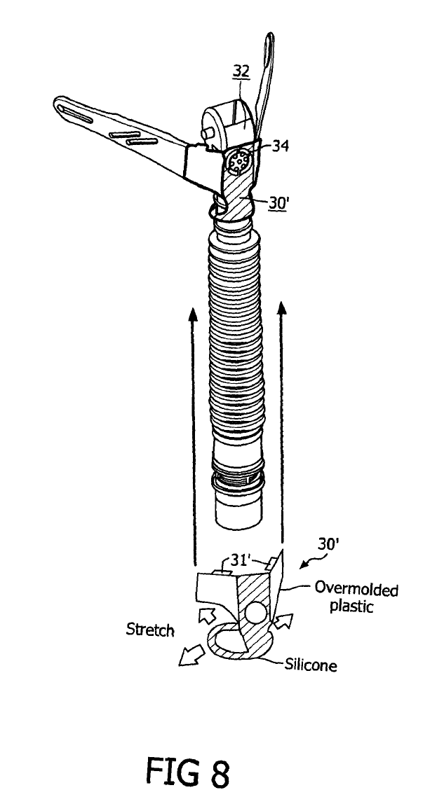

FIG. 8 is an isometric view of a further deflector mechanism according to an embodiment of the invention shown both uninstalled and installed on a portion of a patient interface device.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

A system 2 adapted to provide a regimen of respiratory therapy to a patient is generally shown in FIG. 1. System 2 includes pressure generating device 4, patient circuit 6, patient interface device 8, and an exhaust port 10 included on an elbow 11 along patient circuit 6. Although system 2 is discussed as including pressure generating device 4, patient circuit 6, and patient interface device 8, it is contemplated that other systems may be employed while remaining within the scope of the present invention. For example, and without limitation, a system in which the pressure generating device is coupled to a patient interface device having an integrated exhaust port 10 is contemplated.

Pressure generating device 4 is structured to generate a flow of breathing gas and may include, without limitation, ventilators, constant pressure support devices (such as a continuous positive airway pressure device, or CPAP device), variable pressure devices (e.g., BiPAp®, Bi-Flex®, or C-Flex™ devices manufactured and distributed by Philips Respironics of Murrysville, Pa.), and auto-titration pressure support devices.

Patient circuit 6 is structured to communicate the flow of breathing gas from pressure generating device 4 to patient interface device 8. Typically, patient circuit 6 includes a conduit or tube that couples pressure generating device 4 and patient interface device 8. In the current embodiment, conduit 6 includes an elbow 11 coupled to the interface device 8 which includes exhaust port 10 which allows for the venting of exhaust gases 12 therefrom.

Patient interface device 8 is typically a nasal or nasal/oral mask structured to be placed on and/or over the face of a patient. Any type of patient interface device 8, however, which facilitates the delivery of the flow of breathing gas to, and the removal of a flow of exhalation gas from, the airway of such a patient may be used while remaining within the scope of the present invention. In the example shown in FIG. 1, patient interface device 8 includes cushion 8a, rigid shell 8b, and forehead support 8c. A headgear having straps (not shown) may be attached to shell 8b and forehead support 8c to secure patient interface device 8 to the patient's head.

An opening in shell 8b, to which exhaust elbow 11 is coupled, allows the flow of breathing gas from pressure generating device 4 to be communicated to an interior space defined by shell 8b and cushion 8a, and then, to the airway of a patient. The opening in shell 8b also allows the flow of exhalation gas (from the airway of such a patient) to be communicated to elbow 11 and exhaust port 10 in the current embodiment. Although illustrated in a separate elbow component 11 in FIG. 1, it is contemplated that exhaust port 10 may be incorporated into, for example and without limitation, patient interface 8 and/or different variations of patient circuit 6 while remaining within the scope of the present invention.

FIG. 2 shows an improved system 2′ in accordance with an embodiment of the present invention. System 2′ includes the same components as system 2 discussed above along with a deflector mechanism 20, in accordance with an embodiment of the present invention, positioned on elbow 11 generally covering exhaust port 10. Preferably deflector mechanism 20 is formed of a rigid and/or semi-rigid material. In an exemplary embodiment, deflector mechanism 20 is made in a rigid material with a low surface-friction coefficient to maximize the flow pattern. It is to be appreciated that a semi-rigid material may be used for ease of assembly. Referring to FIGS. 3 and 4, deflector mechanism 20 includes a mounting portion 22 and a deflector portion 24. Mounting portion 22 is structured to couple deflector mechanism 20 to elbow 11 in a manner such that deflector portion 24 is generally disposed at or about exhaust port 10. Such coupling is accomplished via a snap fit or other suitable attachment method that readily provides for deflector mechanism 20 to be retrofit to known systems 2.

In an exemplary embodiment, mounting portion 22 is further structured to clamp tight to elbow 11 such that a tight seal between mounting portion 22 and elbow 11 is formed. Optionally, a seal member 26 (FIGS. 3 and 4) may be provided between mounting portion 22 and elbow 11 to help ensure a tight seal between mounting portion 22 and elbow 11. It is to be appreciated that the structure of mounting portion 22 may be varied to accommodate the structure of the region where exhaust port 10 is disposed in order to provide for the coupling of deflector portion 24 relative to the exhaust port 10 as discussed further below.

Deflector portion 24 generally serves a dual purpose in regard to the handling of exhaust gases 12 exiting from exhaust port 10. First, as will be evident in the examples described herein, deflector portion 24 provides for the redirection of exhaust gases 12 in a direction that would tend to be less noticeable to a patient wearing patient interface 8 as well as others around the patient (e.g., without limitation, downward along patient circuit 6 as shown in FIG. 2). Second, the noise gain resulting from the flow of exhaust gases 12 may be adjusted to a level desirable to the patient and others nearby by altering a characteristic of deflector portion 24, such as the geometry and/or surface characteristics of deflector portion 24.

Referring to FIG. 4, an example deflector mechanism 20 according to an embodiment of the invention is shown in which the inner surface 25 of deflector portion 24 is spaced a distance D from exhaust port 10 when deflector mechanism 20 is mounted on elbow 11 via mounting portion 22. Such positioning of surface 25 of deflector portion 24 provides for deflector mechanism 20 to selectively direct the flow of exhaust gases 12 exiting exhaust port 10 without noticeably restricting the flow of such exhaust gases from exhaust port 10. Generally, the distance D is chosen to be a value that results in less than a 5% increase of flow resistance from exhaust port 10 as such increase is generally negligible and not noticeable to a patient. In other words, the pressure drop for the flow of gas exiting elbow 11 into the area between elbow 11 and deflector 20 is greater the pressure drop for the flow of gas exiting the area between elbow 11 and deflector 20 to ambient atmosphere. Thus, the addition of deflector mechanism 20 adds no additional resistance to the exhaust gas flow. In this manner, the deflector mechanism is structured to selectively direct the exhaust gases without an addition additional restriction, i.e., without adding another pressure drop, to the flow of the exhaust gases.

By altering the spacing D between surface 25 of deflector portion 24 and exhaust port 10, the sound of the exhaust gases 12 exiting exhaust port 10 may be modified in a manner that is less bothersome to a patient or others nearby. Such modification may include, for example, without limitation, dampening the sound of exiting exhaust gases 12 or causing the sound of exiting exhaust gases 12 to mimic the sound of white noise.

FIG. 5 shows another example of a deflector mechanism 20′ according to an embodiment of the invention similar to that shown in FIG. 4 in which a surface characteristic 28′ has been modified/added to surface 25′ of deflector portion 24′ in order to alter an attribute of the noise gain of exhaust gases 12 exiting exhaust port 10. Such surface characteristic 28′ may include one or more of: (a) a particular surface finish or finishes provided directly in the material from which deflector portion 24′ is formed (e.g., without limitation, smooth finish, rough finish, ridges, bumps, grooves) and (b) a material different from the deflector portion 24′ added to surface 25′ having desired physical properties or texture (e.g., without limitation, material with a different surface finish, materials with a different coefficient of friction, ridges, bumps, grooves).

Referring to FIG. 6, a deflector mechanism 20″ in accordance with another embodiment of the invention is shown. Like deflector mechanisms 20 and 20′ previously described, deflector mechanism 20′ includes a mounting portion 22″ and a deflector portion 24″, with mounting portion 22″ being structured to couple deflector mechanism 20″ to elbow 11 such that deflector portion 24″ is positioned at or about exhaust port 10. However, unlike the embodiment previously described, deflector portion 24″ further includes a diaphragm member 26″ generally positioned above exhaust port 10. In an exemplary embodiment, diaphragm member 26″ is formed of silicone or other suitable flexible material bonded to the surrounding rigid or semi-rigid material (previously discussed) which forms the remainder of deflector and mounting portions 24″, 22″.

Flexible diaphragm member 26″ modifies the sound of exiting exhaust gases 12 by providing a changing spacing proportional to the pressure of the exiting exhaust gases 12. The changing spacing is controlled by the physical properties of the material and the wall thickness t of flexible diaphragm member 26″ to provide a desired “spring” force to counter the flow of exiting exhaust gases 12. The use of such flexible diaphragm member 26″ provides for a controlled diversion of the flow of exiting exhaust gases 12 in a different manner from the previously described embodiment which utilized a fixed spacing. A fixed spacing generally acts like an orifice, which has a fixed proportion between the flow and pressure of exhaust gases 12 passing through. In embodiments employing a flexible diaphragm, the spacing changes with the pressure (i.e., spacing increases with pressure to provide a larger spacing at higher pressure hence higher flow which serves to reduce the pressure drop to ensure there is no added restriction to the flow). The changing spacing also provide a damping effect which additionally helps to lower the noise produced by the exiting exhaust gases 12 and helps to stabilize the flow.

FIG. 7 depicts another embodiment of the invention which utilizes a mounting member 30 which is selectively attachable to a portion of patient interface device 32 at or about the exhaust port 34. Such selective attachment may be accomplished through the use of a number of mounting clips 31, which can generally snap onto a portion of the patient interface device 32. Mounting member 30 includes an opening 36 to which a deflector mechanism 38 of similar design and functionality as deflector mechanisms 20, 20′, and 20″ may be either fixed or removably coupled such that deflector mechanism 38 selectively directs and modifies the flow of exhaust gases 12 exiting exhaust port 34 in a manner as previously discussed in regard to deflector mechanisms 20, 20′, and 20″.

FIG. 8 depicts another embodiment of the invention similar to that shown in FIG. 7 in which a mounting member 30′ (shown both installed on, and removed from, patient interface device 32) is utilized for mounting a deflector mechanism (not shown) about exhaust port 34. Like mounting member 30, mounting member 30′ also includes an opening 36 to which a deflector mechanism 38 of similar design and functionality as deflector mechanisms 20, 20′ and 20″ may be either fixed or removably coupled. Also similar to the embodiment shown in FIG. 7, mounting member 30′ utilizes a number of mounting clips 31′ which can generally snap onto a portion of the patient interface device 32. Mounting member 30′ further employs an elastic portion 33′ in addition to mounting clips 31′ to allow for selective coupling of mounting member 30′, and thus the deflector mechanism 38 coupled thereto, to the patient interface device 32 about exhaust port 34.

Accordingly, it is to be readily appreciated that mounting members 30 and 30′ help to provide for the mounting of a deflector mechanism, according to any of the embodiments disclosed or implied herein, to a patient interface device at or about an exhaust port.

It can be further appreciated that the present invention provides an improved apparatus and method for selectively manipulating the flow of exhaust gas expelled from a patient interface device such that the exhaust gas is expelled in a manner that does not interfere with the patient and/or the patient's sleep partner. Additionally, the present invention provides an apparatus and method for providing an improved gas port that reduces the noise generated by venting exhaust gases to the atmosphere.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1-8. (canceled)

9. A respiratory interface device comprising:
(a) a cushion;
(b) a mask frame supporting the cushion;
(c) a patient circuit coupled to the mask frame adapted to carry a flow of gas;
(d) an exhaust port integrated in one of the mask frame or patient circuit, the exhaust port being structured to vent a flow of exhaust gases from the respiratory interface device, and wherein a first pressure drop is provided for the flow of gas passing through the exhaust port; and
(e) a deflector mechanism comprising:
(1) a deflector portion having a surface, and
(2) a mounting portion coupling the deflector mechanism to the one of the mask frame and the patient circuit at or near the exhaust port such that a cavity is provided between the deflector mechanism and the associated mask frame or the patient circuit, wherein the surface of the deflector portion is structured to selectively direct the flow of exhaust gases to ambient atmosphere such that a second pressure drop is provided for the flow of from the cavity to ambient atmosphere, and wherein the first ressure dro is t reater than the second pressure drop.

10. The respiratory interface device of claim 9, wherein the deflector mechanism includes a predetermined finish on the surface of the deflector portion.

11. The respiratory interface device of claim 10, wherein the predetermined finish is integral with the surface of the deflector portion.

12. The respiratory interface device of claim 10, wherein the predetermined finish comprises a coating applied to the surface of the deflector portion.

13. The respiratory interface device of claim 9, wherein a predetermined spacing (D) is provided between the surface of the deflector portion and the exhaust port.

14. The respiratory interface device of claim 9, wherein the deflector portion is structured to direct the exhaust gases generally along the patient circuit

15. The respiratory interface device of claim 9, wherein the mounting portion further comprises a seal member which sealingly engages the one of the mask frame and patient circuit.

16. The respiratory interface device of claim 9, wherein the deflector mechanism includes a flexible diaphragm member integrated into the deflector portion.

17. (canceled)
